(19) United States
(12) Patent Application Publication
(10) Pub. No.: US 2024/0081675 A1
(43) Pub. Date: Mar. 14, 2024

(54) BREATH BIOMARKERS OF SARS-COV-2 IN CHILDREN AND METHODS OF USE THEREOF FOR THE DIAGNOSIS AND TREATMENT OF COVID-19

(71) Applicant: THE CHILDREN'S HOSPITAL OF PHILADELPHIA, Philadelphia, PA (US)

(72) Inventors: Audrey Ragan ODOM JOHN, Swarthmore, PA (US); Amalia Zoraida BERNA PEREZ, Media, PA (US); Elikplim AKAHO, Philadelphia, PA (US)

(73) Assignee: THE CHILDREN'S HOSPITAL OF PHILADELPHIA, Philadelphia, PA (US)

(21) Appl. No.: 18/251,075

(22) PCT Filed: Nov. 1, 2021
(86) PCT No.: PCT/US2021/057579

§ 371 Date: Apr. 28, 2023

Related U.S. Application Data
(60) Provisional application No. 63/107,891, filed on Oct. 30, 2020.

(51) Int. Cl. A61B 5/08; G01N 33/497

(52) U.S. Cl. CPC A61B 5/082; G01N 33/497; A61B 2560/0431; A61B 2010/0087

(57) ABSTRACT

Compositions and methods for the diagnosis and treatment of Covid 19 are disclosed.

### CROSS-REFERENCE TO RELATED APPLICATION

This application claims priority of U.S. Provisional Application No. 63/107,891 filed Oct. 30, 2020, the entire disclosure being incorporated herein by reference as though set forth in full.

### FIELD OF THE INVENTION

The present invention relates to various methods of diagnosing and treating a subject infected with SARS-CoV-2.

### BACKGROUND OF THE INVENTION

Several publications and patent documents are cited through the specification in order to describe the state of the art to which this invention pertains. Each of these citations is incorporated herein by reference as though set forth in full.

The current COVID-19 pandemic has stressed the threat that viruses pose to the population and the limitations of the current virology testing. The most common strategy to identify acute infection relies on collecting samples either the upper (most commonly nasopharyngeal (NP) swabs) or lower respiratory tract (e.g. bronchoalveolar lavage). The genetic material extracted from these samples is then amplified by RT-PCR. Such testing is expensive, requires multiple reagents for which supplies are limited (swabs, viral transport media, and RNA extraction kits), and necessitates specialized laboratory equipment and trained personnel. Furthermore, concern has been raised about the false negative rate of RT-PCR and the sample techniques. A recent systematic review on false negative concluded that up to 54% of patients could have an initial false negative RT-PCR results [1]. False-negative cases have important implications for isolation and risk of transmission of infected people and for management of the disease.

SARS-CoV-2 infection in adults can lead to an uncontrolled systemic inflammatory reaction (a cytokine storm). This can cause acute respiratory distress syndrome, organ failure and lead to the decease of the patient. Children are less likely to develop severe disease from COVID-19 compared to adults [2], although the reasons for this remain unclear. Despite the fewer cases reported in children, there are concerns about asymptomatic or mild symptomatic pediatrics cases going undetected and unknowing transmitting SARS-CoV-2 in the community. A quick, sensitive, non-invasive technique is urgently needed for the early detection of SARS-CoV-2.

### SUMMARY OF THE INVENTION

In accordance with the present invention, a method for diagnosing or monitoring a subject with a SARS-CoV-2 infection is provided. In one embodiment, the method comprises analyzing a sample of exhaled breath or condensate breath obtained from the subject for elevated levels of volatile organic compounds (VOCs) octanal, nonanal, and heptanal relative to levels observed in uninfected control subjects, wherein said elevated levels are indicative of SARS-CoV-2 infection. The subject may be an adult, adolescent or pediatric subject. Preferably, the subject is a pediatric subject.

In another embodiment, octanal, nonanal, heptanal, decane and/or dodecane, 2-penthyl furan, and optionally tridecane levels are determined. Analysis of VOC is performed using least one technique selected from the group consisting of photo ionization detection, flame ionization detection, gas chromatography—mass spectrometry (GC-MS), proton transfer reaction mass spectrometry (PTR-MS), colorimetry, infrared spectroscopy, electrochemical fuel cell sensing, semiconductor gas sensing, quartz tuning fork (QTF) sensors, electronic noses and combinations thereof. In a preferred embodiment, analysis of the VOCs is conducted using a portable, hand-held breathalyzer device.

In certain aspects, the method of analyzing the sample of exhaled breath or condensate breath obtained from the subject for a series of volatile organic compounds (VOCs) comprising: i) octanal; ii) nonanal; iii) heptanal; iv) decane; v) 2-penthyl furan; and vi) tridecane entails determining a concentration for each of the VOCs; and calculating a cumulative abundance based on the concentrations for the VOCs, wherein the cumulative abundance and the concentration of the VOCs indicates a SARS-CoV-2 infection. In other aspects one or more of levels of the additional VOCs in Table 1A are determined.

The invention also provides a method for diagnosing or monitoring a subject with a SARS-CoV-2 infection, comprising: analyzing a sample of exhaled breath or condensate breath obtained from the subject for a series of volatile organic compounds (VOCs) selected from i) octanal; ii) nonanal; iii) heptanal; iv) decane; v) 2-penthyl furan; and vi) tridecane, comprising: determining a concentration for each of the VOCs; and calculating a cumulative abundance based on the concentrations for the VOCs, wherein the cumulative abundance indicates a SARS-Co-V-2 infection.

In yet another embodiment, a method of detecting a combination of VOCs in a subject selected from octanal, nonanal, heptanal, decane, 2-penthyl furan; and tridecane is disclosed. An exemplary method comprises analyzing a sample of exhaled breath or condensate breath obtained from the subject for said VOCs. In certain aspects of the methods described above, the methods can further comprise condensing or concentrating the sample before analysis.

The invention also provides administering to the subject a pharmaceutical composition comprising a therapeutically effective amount of at least one compound effective against a SARS-CoV-2 infection. Agents to be administered include, without limitation, at least one compound effective against the SARS-CoV-2 infection selected from convalescent plasma, anti-virals, remdesivir, monoclonal antibodies or antibody cocktails or combinations, soluble ACE-2 and steroids, and combinations thereof.

### BRIEF DESCRIPTION OF THE DRAWINGS

FIGS. 1A-1B. FIG. 1A: Breath collection system for children. Child places mouthpiece (1) between the lips, exhales and volatiles are transferred from two-way valve (2) to SamplePro FlexFilm sample bag (3). FIG. 1B: VOC biomarkers selected as best discriminants between SARS-Cov-2 positive and control (SARS-Cov-2 negative) patients, together with analytical characteristics of each compound.

FIG. 2. Workflow of data analysis and statistics used to create a final predictive model to discriminate SARS-CoV-2-infected from uninfected subjects.

FIGS. 3A-3B. Isoprene is significantly more abundant in breath samples compared to room air. Isoprene is significantly more abundant in breath samples compared to room air. Discovery cohort (FIG. 3A) and Validation cohort (FIG. 3B)]. Higher levels of isoprene were found in breath compared room air in both SARS-CoV-2-infected and -uninfected breath samples. Median with SEM are shown. Note that the sensitivity of the instrument changed between the time of analysis of each cohort, as a result of system maintenance and recalibration.

FIGS. 4A-4B. Biomarkers of SARS-CoV-2 in breath. (FIG. 4A) Volcano plot of breath metabolites. Fold change=(mean abundance SARS-CoV-2 infected)/(mean abundance uninfected). Purple, breath metabolites significantly different (p<0.05) in SARS-CoV-2 infected subjects compared to uninfected. Metabolite identities are shown in heat map. (FIG. 4B) Heat map visualizing abundance of breath biomarkers (presented as z-scores) in a discovery cohort of uninfected- and SARS-CoV-2 infected pediatric subjects.

FIGS. 5A-5B. Pediatric SARS-CoV-2 infection is associated with an increased abundance of breath aldehydes. (FIG. 5A) Three-dimensional GCxGC ToF-MS surface plots of two representative pediatric breath samples (top, SARS-CoV-2 infected; bottom, SARS-CoV-2 uninfected), demonstrating increased abundance of characteristic medium-chain aldehydes (†, heptanal; ‡, octanal; *, nonanal) associated with SARS-CoV-2 infection; 1tR, retention time in minutes; 2tR, retention time in seconds. (FIG. 5B) Scatter plot of breath abundance of candidate SARS-CoV-2 aldehyde biomarkers in uninfected and infected children. Octanal and heptanal were also previously found to be increased in abundance in the breath of adults with COVID-19.15 Median and quartiles are shown. P-values (t tests) are shown for each comparison.

FIGS. 6A-6C. Breath abundance of candidate SARS-CoV-2 biomarkers in the breath of uninfected and infected children (discovery cohort). Median and quartiles are shown. P-values from t-tests are shown for each comparison. FIG. 6A—Dodecane. FIG. 6B—2-pentylfuran. FIG. 6C—Tridecane.

FIG. 7. Breath abundance of candidate SARS-CoV-2-associated biomarkers are not significantly different between febrile and afebrile SARS-CoV-2 patients (discovery cohort). Median with SEM are shown. Adjusted p value (t-test) for all comparisons >0.99.

FIG. 8. Cumulative abundance of breath biomarkers is not associated with cycle threshold (Ct) in SARS-CoV-2-positive patients (Pearson's, r2=0.08).

FIGS. 9A-9F. Breath abundance of candidate SARS-CoV-2 biomarkers in uninfected and infected children in the validation cohort. Median and quartiles are shown. P-values (t-tests) are shown for each comparison. FIG. 9A—Heptanal. FIG. 9B—Octanal. FIG. 9C—Nonanal. FIG. 9D—Dodecane. FIG. 9E—Furan, 2-pentyl. FIG. 9F—Tridecane.

FIG. 10. Discriminatory power of candidate SARS-CoV-2 breath biomarkers in an independent cohort of children. Principal component analysis was performed with six candidate biomarkers of SARS-CoV-2 using breath samples from an independent validation cohort of children with and without SARS-CoV-2 infection.

FIGS. 11A-11C. Performance characteristics of a cumulative abundance metric for SARS-CoV-2 diagnosis. (FIG. 11A) The cumulative abundance (normalized to internal standard) of six candidate biomarkers readily distinguishes breath profiles from an independent validation cohort of children with and without SARS-CoV-2 infection (t test, p=0.001). The cumulative abundance is the sum of abundances of the six candidate biomarkers. (FIG. 11B) Distribution of cumulative abundance of biomarkers from SARS-CoV-2 infected and uninfected children. Red line, threshold of discrimination between infected and uninfected. (FIG. 11C) Receiver operator characteristics (ROC) curve for the cumulative abundance of 6 biomarkers. Dotted line indicates expected results if predictive power is no better than random chance. Using threshold, this cumulative abundance metric yields 91% sensitivity and 75% specificity.

FIG. 12. Ketones associated with adult COVID-19 are not enriched in the breath of children with SARS-CoV-2 infection. Acetone and 2-butanone were previously reported to be enriched in the breath of adults with COVID-19.¹⁵ No significant differences were found between SARS-CoV-2 infected and uninfected pediatric samples in the pilot data (shown). Similar results were observed in an independent validation cohort (FIG. 9). Median and quartiles are shown. P-values (t tests) are shown for each comparison.

### DETAILED DESCRIPTION OF THE INVENTION

Upper respiratory and pulmonary infections are known to alter host metabolism, resulting in distinct volatile organic compounds (VOCs) present in breath exhalate. Indeed, a wide variety of pulmonary infections are known to alter breath metabolite profiles in humans, including Mycobacterium tuberculosis [3], Aspergillus spp., and ventilator-associated pneumonia [4]. Respiratory viral infections also impact volatile production in vitro and in vivo. For example, infection with either respiratory rhinovirus [5] or influenza in cell culture [6] results in characteristic and reproducible changes in volatiles released. Studies of breath volatiles in influenza-infected pigs demonstrates an increase of acetaldehyde, propanal and n-propyl acetate [7], confirming that host metabolic changes induced by respiratory viral infection can be detected in breath.

Since SARS-Cov-2 cellular infection simultaneously affects many signal transduction and protein expression pathways, which differs from influenza and rhinovirus, a large and unique downstream VOC production can be expected [8]. Moreover the immune response for the control of SARS-CoV-2 infection triggers also a cascade of molecules inducing a cytokine storm, which leads to lung injury, causing destruction of the cellular structure due to oxidative stress in the body which might release VOCs into breath. There is also evidence that viral respiratory infections could cause alterations in both respiratory and gastro-intestinal microbiome [9]. These microbiome alterations are likely to cause metabolic changes that could be detectable in either the breath or stool [10]. To date, there is strong evidence that there is a unique VOC profile associated with COVID-19 infection and dogs can be trained to recognize it from saliva/tracheal samples [11] and sweat samples [12].

COVID-19 control efforts have been hampered by transmission from pre-symptomatic individuals infected with SARS-CoV2. Prolonged asymptomatic respiratory viral shedding in children has been described and appears to be another important reservoir for ongoing transmission. The current standard diagnostic strategy to identify SARS-CoV2 infection relies on qPCR of specific viral sequences from respiratory samples, which is expensive, uncomfortable, and relatively slow. A rapid non-invasive method to detect asymptomatic or mildly symptomatic infection would have a major impact on public health campaigns to control COVID-19.

We hypothesized that candidate biomarkers characterize the exhaled breath of children with SARS-CoV2 infection. To test this hypothesis, we enrolled SARS-CoV-2-infected and uninfected children admitted to a major pediatric academic medical center and analyzed their breath volatile composition. Targeted volatiles analysis revealed that six volatile organic compounds increased significantly in SARS-CoV-2-infected children. Three candidate biomarkers (octanal, nonanal, and heptanal) drew especial attention, because viral infections have previously been shown to induce aldehyde production. Together, these biomarkers demonstrated 100% sensitivity and 66.6% specificity. Using these data, we have developed a “breathalzyer” test for SARS-CoV-2 infection in children.

The present invention includes methods for diagnosing or monitoring a subject with a Sars-2 infection (Covid-19). In some embodiments, the methods comprise analyzing a sample of exhaled breath or condensate breath obtained from the subject for the series of VOCs described herein, wherein the concentration of the VOCs indicates a SARS-CoV-2 infection.

In various embodiments, the sample is analyzed for VOCs comprising octanal, nonanal, heptanal, decane and/or dodecane, 2-penthyl furan; and tridecane.

In various embodiments, methods for analyzing a sample of exhaled breath or condensate breath obtained from a subject can include the use of at least one technique selected from the group consisting of photo ionization detection, flame ionization detection, gas chromatography—mass spectrometry (GC-MS), proton transfer reaction mass spectrometry (PTR-MS), colorimetry, infrared spectroscopy, electrochemical fuel cell sensing, semiconductor gas sensing, quartz tuning fork (QTF) sensors, electronic noses and combinations thereof. These methods are described in more detail herein.

In certain embodiments, the method comprises use of an electronic nose, or any microarray capable of sensing multiple volatile signatures, particularly one calibrated to the detection of volatile organic compounds (Chang et al, Science Reports, 6(2016): 23970). In further embodiments, the method comprises use of a portable wireless volatile organic compound monitoring device that employs quartz tuning fork (QTF) sensors (Deng et al. Sensors 2016, 16(12), 2060). These techniques involve adsorption of VOCs onto modified (coated) QTFs which alters their resonance frequency and enables quantification of VOC concentration.

The analysis described for the methods herein could also include use of a portable device comprising a sample collection and pre-concentration unit, a sample separation column, and a sensitive, selective and fast sensor (IEEE Sens J. 2013 May; 13(5): 1748-1755).

In some embodiments, the method further comprises the use of solid-phase micro-extraction fibers to extract and concentrate volatile chemicals in exhaled breath for further analysis. For example, various methods can include the use of micro-extraction fibers alongside GC-MS to detect VOCs in exhaled breath of human patients (Gao et al, J. Breath Res. 10:2 (2016) 027102). In further embodiments, the method comprises use of PTR mass spectrometry to detect VOCs in collected breath of subjects (O'Hara et al., J. Breath Res. 10:4 (2016)). PTR mass spectrometry uses gas phase hydronium (H₃0+) ions to ionize trace VOCs in an air sample in order to detect and identify them using mass spectrometry. In still other embodiments, the method comprises use of fast gas chromatography—flame ionization detection (Fast-GC-FID) which is known in the art to detect VOCs in ambient air samples (Jones et al, Atmos. Meas. Tech, 7, 1259-1275, 2014). Briefly, this method involves separating volatile chemicals on a gas column and using a hydrogen flame to oxidize them for detection.

In various embodiments, the analysis of the VOCs listed above is conducted using a portable, hand-held breathalyzer or electronic nose device. The technique or device used for analysis can also include a display or be in communication with a further device (e.g., monitor or printer) that displays the results of the analysis.

Further the methods for diagnosing or monitoring a subject with SARS-CoV-2 infection (COVID-19) comprise analyzing a sample of exhaled breath obtained from the subject for a series of volatile organic compounds (VOCs) comprising: octanal, nonanal, heptanal, decane or dodecane, 2-pentyl furan, and tridecane; determining a concentration for each of the VOCs; and calculating a cumulative abundance based on the concentrations for the VOCs, wherein the cumulative abundance indicates SARS-CoV-2 infection. Parameters used for the analysis of these compounds are further specified in Table 1A.

In certain embodiments, concentrations of the series of VOCs in a subject are compared to concentrations in a healthy individual.

In additional embodiments, the methods of diagnosing or monitoring a SARS-CoV-2 infection can comprise the combination of any of the methods described herein.

In some embodiments of the present invention, the methods of diagnosing or monitoring a SARS-CoV-2 infection by analyzing breath samples for a series of VOCs can further comprise analyzing the same sample for one or more volatile organic chemicals selected from those listed in Table 1A and combinations thereof. Analyzing for a combination of biomarkers of the SARS-CoV-2 infection can enhance the effectiveness and accuracy of the diagnostic/monitoring methods described herein.

In various embodiments of diagnosis and monitoring of subjects using the methods described herein, samples from the subjects can be exhaled breath or condensate breath. In various embodiments, the sample obtained from the subject is exhaled breath. In some embodiments, the method further comprises condensing or concentrating the sample before analysis.

In various embodiments, the concentration of the series of VOCs in breath or breath condensate aids in the diagnosis or the monitoring of a SARS-Cov-2 infection. In some embodiments, the concentration of the series of VOCs in the breath or breath condensate is compared to levels of the series of VOCs in the breath of other subjects determined to be free of SARS-CoV-2 infection (e.g., baseline VOC concentrations).

The current invention is further directed to methods of detecting select volatile organic chemicals in samples of exhaled breath or condensed breath from a subject. In some embodiments, the methods comprise detecting of at least one VOC in a subject by analyzing a sample of exhaled breath or condensed breath obtained from the subject for as described herein. In other embodiments, the methods comprise detecting a series of volatile organic chemicals in a subject and determining a concentration for each of the VOCs. The series of VOCs can include comprising octanal, nonanal, heptanal, decane or dodecane, 2-penthyl furan; and tridecane. In further embodiments, the methods of detecting volatile organic chemicals in a sample comprise the combination of any of the methods described herein. Specifically, various embodiment of the invention comprise detecting the series of VOCs as described herein. Other VOCs to be detected include without limitation, the additional VOCs listed in Table 1A.

In the methods described herein, the sample obtained from the subject is exhaled breath. In some embodiments, the methods further comprise condensing or concentrating the sample before analysis.

In various embodiments, the subject exhibits one or more characteristic symptoms or etiology known to be associated with Covid-19. These include, but are not limited to, high fever, prostration, exhaustion, respiratory distress (acidotic breathing), gastrointestinal distress, cytokine storm syndrome, circulatory collapse, lung damage, kidney damage, neurological deficits, disorientation, headache, chills, aches, coughing, and/or sneezing.

The methods of the present invention can further comprise administering to the subject a pharmaceutical composition comprising a therapeutically effective amount of at least one compound effective for treatment of Covid 19. For example, the compound effective against the SARS-CoV-2 virus can include at least one compound selected from convalescent plasma, anti-viral agents, remdesivir, monoclonal antibodies or antibody cocktails or combinations, soluble ACE-2 and steroids, and combinations thereof.

The following materials and methods are provided to facilitate the practice of the present invention.

### Chemical Standards and Solutions

Nonanal, octanal, heptanal, tridecane, and 2-pentylfuran and isoprene were purchased from Sigma-Aldrich (St Louis, MO, US). Dodecane was purchased from Merck (Darmstadt, Germany). To spike the compound of interest into a sorbent tube, a 10 ppm solution was prepared in HPLC grade methanol. Using a solution loading rig (Markes International Limited, UK), 1 μL of the solution was spiked into a sorbent tube. The sorbent tube was flushed for 3 min with nitrogen at a flow of 100 mL·min⁻¹. All the stock solutions were stored in glass vials and kept at 4° C. Sorbent tubes containing standards were analyzed by GCxGC BenchTOF-MS following the same protocols as described below for breath samples.

### Study Approval and Enrollment

Prior to enrollment, the study was approved by the Children's Hospital of Philadelphia (CHOP) Human Research Ethics Committee (IRB 20-017503) and by the CHOP Institutional Biosafety Committee (IBC 19-000145) for handling of human samples potentially containing SARS-CoV-2. Breath samples were collected from children (4-18 years of age) hospitalized in the Special Isolation Unit at CHOP who had been diagnosed as SARS-CoV-2 positive by nasopharyngeal swab RT-PCR on admission (n=11). Samples from uninfected individuals were obtained from nasopharyngeal RT-PCR-negative subjects enrolled from the Emergency Department Extended Care Unit of the Children's Hospital of Philadelphia (n=15). Samples were collected between June and August 2020. The viral load of patient nasopharyngeal swab samples was estimated by cycle threshold value (Ct-value) of the N gene, with lower Ct-values indicating a higher viral load. Samples were considered positive if the Ct-value was ≤40, and Ct values of positive test results were obtained (Table 1). For validation studies, we collected an additional set of samples from SARS-CoV-2 infected children (n=12) and from SARS-CoV-2 uninfected individuals (n=12). Samples were collected as above from CHOP between October 2020 and March 2021. The sample size for this cohort was based on a calculated effect size of 1.5 between SARS-CoV-2 infected and uninfected samples for the 6 biomarkers, which predicted that 12 samples in each group would yield a power (1−β error prob) of 0.97 (p<0.05).

Exclusion criteria for control subjects included current rhinorrhea, cough, or diarrhea, in order to exclude individuals that may have false negative SARS-CoV-2 testing. In addition, subjects were excluded if they required oxygen supplementation within 3 h of breath sample collection. Samples were not screened for common circulating non-SARS-CoV-2 human coronaviruses or other viral respiratory pathogens.

### Breath Sample Collection

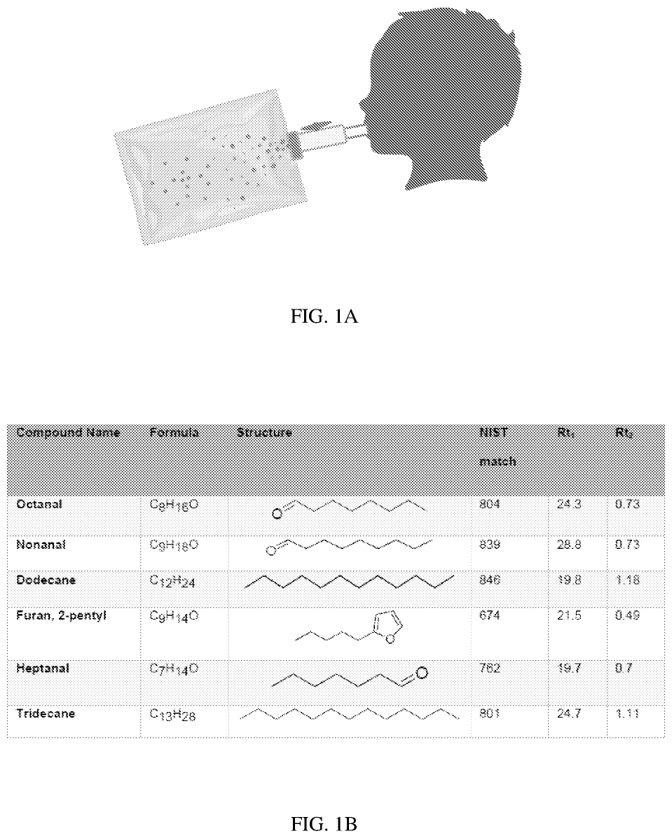

Breath collection was performed as previously described. In brief, SARS-CoV-2 infected and uninfected subjects exhaled through a disposable cardboard mouthpiece connected to a chamber. The chamber was then attached using tubing to a 3-L SamplePro FlexFilm sample bag (SKC Inc., Pennsylvania) (FIG. 1A). The volunteers were asked to take a few deep breaths, place the cardboard tube between the lips, and exhale completely. Neither a nose clip nor VOC filter were used. Breath from the bags was transferred to a sorbent tube as previously described.26,28 Briefly, 1 L of the breath sample was transferred to sorbent tube at 200 mL min-1 using an electric pump, so that all tubes had consistently the same volume. Three-bed Universal sorbent tubes containing Tenax, Carbograph, and Carboxen were used (Markes International Limited, UK). For each participant, ambient air samples and breath samples were collected from the same room. Samples were stored at 4° C. until the time of analysis (within 2 weeks of collection). Samples were analyzed using thermal desorption and GCxGC BenchTOF-MS (SepSolve Analytical, UK). Analytical parameters are described below.

### Thermal Desorption and GCxGC Parameters

Prior to analysis, sorbent tubes were brought to room temperature and loaded into autosampler (Utra-xr, Markes International, UK). A gaseous standard mixture (1.01 ppm Bromochloromethane, 1.04 ppm 1,4-Difluorobenzene, 1.04 ppm Chlorobenzene-D5, 0.96 ppm 4-bromofluorobenzene) was immediately added to each tube, followed by a purge pre-desorption step consisting of 10 min with He at 50 mL*min1, to remove water content in breath samples. Tubes were thermally desorbed for 10 min at 270° C. (Unity-xr, Markes International, UK) and transferred to a “Universal” cold trap which matched the sorbent of the sample tube, held at 10° C. and subsequently heated to 300° C., to minimize band broadening. The split flow after the cold trap was 15 mL*min-1.

Analysis by two-dimensional gas chromatography was conducted using an Agilent 7890B GC system, fitted with a flow modulator and a three-way splitter plate coupled to a flame ionization detector and a time-of-flight mass spectrometer with electron ionization (SepSolve, UK). Chromatographic analysis was performed using a Stabilwax (30 m×250 μm ID×0.25 m df) as the first dimension (1D)-GC column and a Rtx-200 MS (5 m×250 μm ID×0.1 μm df) as second dimension (2D)-GC column, both purchased from Restek (Bellefonte, PA, US). The following GC oven temperature program was used: initial temperature 40° C. and held for 1 min, ramped to 260° C. at 3° C.*min-1. The final temperature of 260° C. was held for 1 min. The total run time for the analysis was 75 min. Helium carrier gas was flowed at a rate of 1.2 mL*min-1. The flow modulator (Insight, SepSolve Analytical, UK) had a loop with dimensions 0.53 mm i.d.×110 mm length (loop volume: 25 uL), and the modulation time was 2 s total.

### TOF-MS Conditions

The GCxGC was interfaced with a BenchTOF-select time-of-flight mass spectrometer (SepSolve Analytical, UK). The acquisition speed was 50 Hz and mass range was 30-400 m/z. The ion source and transfer line were set at 250° C. and 270° C. respectively and filament voltage at 1.6 V. Electron ionization energy was 70 eV. ChromSpace (SepSolve Analytical, UK) was used to synchronize and control the INSIGHT modulator, thermal desorption, GC, and TOF. BenchTOF shows a linear response for VOCs from 500 fg to 10 ng with a limit of detection of <20 fg.

### Data Processing and Statistical Analyses

Data was acquired and processed using ChromSpace (SepSolve Analytical, UK). All statistical analyses were performed using RStudio v1.3.1073 (PBC, Boston, MA) and GraphPad Prism V.8.4.3 (GraphPad Software, San Diego, CA).

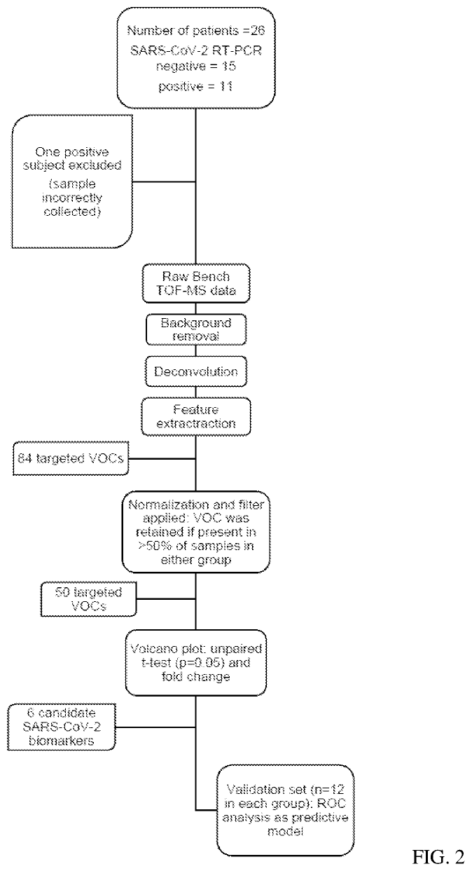

The workflow for data processing and statistical analysis is shown in FIG. 2. Background from the raw BenchTOF data file was removed using the ChromSpace, the Dynamic Background Compensate (DBC) of 0.2 s peak width and noise factor 6.9 for typical GCxGC data was applied. DBC files were integrated, peak detection algorithm used was deconvolution with a minimum ion count of 2000, and peak filters: absolute minimum peak area set at 15,000 counts and absolute minimum peak high 10,000, no relative threshold was set for either mass height or absolute area. Compounds were given annotations using the NIST v.17 reference library. Deconvoluted peaks were exported into .xls format file. The data were then processed using RStudio to generate integrated signal for every isolated feature. Isolated features included 84 targeted volatiles. We targeted volatiles that have been previously associated with respiratory viruses from cell culture, from in vitro airway cells infected with human rhinovirus, in vivo breath profile in swine during Influenza A infection, review on volatiles from the healthy human body [5-7, 15-17], and authors' own unpublished breath VOC library. The data included three internal standards (Table 1A). Data were placed into a 2-dimensional matrix Chromatographic data was first normalized using internal standard (Benzene, 1,4-difluoro-) and a volatile was retained if it was present in more than 50% of the samples in either group (i.e. control or COVID group). In total 50 VOCs were retained and used for further statistical analysis. Unpaired t-test was used to identify metabolites that were significantly different between control groups and COVID-19 groups, with a p-value of 0.05 established as the threshold for statistical significance. Fold change (control/COVID-19 VOC) was also calculated and used to carry out volcano plot. We chose not to perform multiple comparison in this small sample set because an unfortunate byproduct of correcting for multiple comparisons is that we may increase the number of false negatives [18]. In this context false negatives would imply an incorrect diagnose of a potential COVID-19 patient.

Table 1A shows chemical and analytical characteristics of each compound.
a. Compounds in bold are those that met the criteria i.e. present in >50% of samples in either group;
b. Compounds in bold and italic are biomarkers for SARS-CoV-2. Identity was confirmed with reference standards;
c.(*) indicates that compound is an internal standard in the breath sample.

TABLE 1A

| | | | | | Mass | | | |
|---|---|---|---|---|---|---|---|---|
| | | | | Exact | spectral | | | |
| No | Compound_Name | Formula | CAS | mass | match | 1t_{R}/min | 2t_{R}/s | Ref. |
| 1 | 1,3-Butadiene | C4H6 | 106-99-0 | 54.047 | 337 | 24.413 | 0.225 | i |
| 2 | 1-Butanol | C4H10O | 71-36-3 | 74.073 | 826 | 18.089 | 0.225 | i |
| 3 | 1-Butanol, 2- | C6H14O | 97-95-0 | 102.1 | 736 | 8.000 | 0.207 | i |
| | ethyl- | | | | | | | |
| 4 | 1-Butanol, 3- | C5H12O | 123-51-3 | 88.089 | 516 | 22.387 | 0.242 | i |
| | methyl- | | | | | | | |
| 5 | 1-Buten-3-yne | C4H4 | 689-97-4 | 52.031 | 388 | 15.600 | 1.793 | i |
| 6 | 1-Hexanethiol, 2- | C8H18S | 7341-17-5 | 146.11 | 502 | 32.955 | 0.264 | viii |
| | ethyl- | | | | | | | |
| 7 | 1-Hexanol, 2- | C8H18O | 104-76-7 | 130.14 | 896 | 32.642 | 0.280 | i |
| | ethyl- | | | | | | | |
| 8 | 1-Hexene, 3,4- | C8H16 | 16745-94-1 | 112.13 | 573 | 7.549 | 0.311 | i |
| | dimethyl- | | | | | | | |
| 9 | 1H-Pyrrole, 1- | C5H7N | 96-54-8 | 81.058 | 380 | 9.900 | 0.246 | i |
| | methyl- | | | | | | | |
| 10 | 1-Octadecyne | C18H34 | 629-89-0 | 250.27 | 649 | 37.467 | 0.962 | viii |
| 11 | 1-Octanol | C8H18O | 111-87-5 | 130.14 | 704 | 35.417 | 0.255 | i |
| 12 | 1-Penten-3-yne | C5H6 | 646-05-9 | 66.047 | 402 | 15.967 | 0.084 | viii |
| 13 | 1-Propanol | C3H8O | 71-23-8 | 60.058 | 690 | 13.962 | 0.197 | i |
| 14 | 1-Propanol, 3- | C4H10OS | 505-10-2 | 106.05 | 414 | 22.100 | 0.272 | i |
| | (methylthio)- | | | | | | | |
| 15 | 2,4-Hexadiyne- | C6H6O2 | 3031-68-3 | 110.04 | 376 | 1.033 | 0.154 | viii |
| | 1,6-diol | | | | | | | |
| 16 | 2,4-Pentanediol | C5H12O2 | 625-69-4 | 104.08 | 375 | 55.712 | 0.363 | viii |
| 17 | 2-Butanone | C4H8O | 78-93-3 | 72.058 | 681 | 10.044 | 0.425 | ii |
| 18 | 2-Dodecanol | C12H26O | 10203-28-8 | 186.2 | 438 | 67.167 | 0.701 | viii |
| 19 | 2-Dodecanone | C12H24O | 6175-49-1 | 184.18 | 427 | 73.100 | 0.688 | i |
| 20 | 2-Dodecenal | C12H22O | 4826-62-4 | 182.17 | 659 | 17.648 | 1.063 | viii |
| 21 | 2-Pentanol, 4- | C6H14O | 108-11-2 | 102.1 | 412 | 56.481 | 0.145 | viii |
| | methyl- | | | | | | | |
| 22 | 2-Propanone, | C7H14O3 | 5774-26-5 | 146.09 | 375 | 45.397 | 0.355 | viii |
| | 1,1-diethoxy- | | | | | | | |
| 23 | 2-Propenal | C3H4O | 107-02-8 | 56.026 | 662 | 9.026 | 0.277 | ii |
| 24 | 3-Hexanol | C6H14O | 623-37-0 | 102.1 | 408 | 73.752 | 0.288 | viii |
| 25 | 3- | C5H6N2 | 1632-76-4 | 94.053 | 374 | 15.833 | 0.080 | viii |
| | Methylpyridazine | | | | | | | |
| 26 | Acetic acid, | C3H6O2S | 2365-48-2 | 106.01 | 373 | 47.767 | 0.299 | viii |
| | mercapto-, | | | | | | | |
| | methyl ester | | | | | | | |
| 27 | Acetic acid, | C3H6O3 | 625-45-6 | 90.032 | 451 | 12.067 | 0.537 | viii |
| | methoxy- | | | | | | | |
| 28 | Acetone | C3H6O | 67-64-1 | 58.042 | 813 | 8.537 | 0.329 | ii, iv, |
| | | | | | | | | vi |
| 29 | Benzaldehyde | C7H6O | 100-52-7 | 106.04 | 884 | 34.708 | 0.305 | ii |
| 30 | Benzene | C6H6 | 71-43-2 | 78.047 | 901 | 11.046 | 0.301 | i, ii |
| 31 | Benzene, 1,4 | C6H4F2 | 540-36-3 | 114.03 | 900 | 13.525 | 0.313 | |
| | difluoro- (*) | | | | | | | |
| 32 | Benzene, 1- | C6H4BrF | 460-00-4 | 173.95 | 882 | 27.155 | 0.309 | |
| | bromo-4-fluoro- | | | | | | | |
| | (*) | | | | | | | |
| 33 | Benzeneacetalde | C8H8O | 122-78-1 | 120.06 | 621 | 20.684 | 0.487 | viii |
| | hyde | | | | | | | |
| 34 | Benzoic acid | C7H6O2 | 65-85-0 | 122.04 | 623 | 65.585 | 0.017 | i |
| 35 | Benzyl alcohol | C7H8O | 100-51-6 | 108.06 | 855 | 47.687 | 0.109 | i |
| 36 | Butanal | C4H8O | 123-72-8 | 72.058 | 642 | 9.545 | 0.378 | i |
| 37 | Butane, 2,3- | C6H14 | 79-29-8 | 86.11 | 736 | 6.815 | 0.230 | i |
| | dimethyl- | | | | | | | |
| 38 | Butanoic acid | C4H8O2 | 107-92-6 | 88.052 | 627 | 38.522 | 0.084 | i |
| 39 | Butyl lactate | C7H14O3 | 138-22-7 | 146.09 | 352 | 63.500 | 1.060 | viii |
| 40 | Camphene | C10H16 | 79-92-5 | 136.13 | 776 | 14.700 | 0.698 | i |
| 41 | Chlorobenzene- | C6ClD5 | 3114-55-4 | 117.04 | 759 | 21.020 | 0.342 | |
| | d5 (*) | | | | | | | |
| 42 | Decane | C10H22 | 124-18-5 | 142.17 | 743 | 12.486 | 0.906 | i, ii |
| 43 | Dimethyl sulfide | C2H6S | 75-18-3 | 62.019 | 390 | 68.715 | 0.629 | iv |
| 44 | Dimethylamine | C2H7N | 124-40-3 | 45.058 | 561 | 7.267 | 0.196 | vii |
| 45 | 2,3-Butanediol | C4H10O2 | 6982-25-8 | 90.068 | 526 | 58.167 | 0.008 | i |
| 46 | D-Limonene | C10H16 | 5989-27-5 | 136.13 | 884 | 20.049 | 0.581 | i |
| 47 | [symbol] | C12H26 | 112-40-3 | 170.2 | 846 | 19.811 | 1.182 | ii |
| 48 | Ethanol, 2- | C2H5BrO | 540-51-2 | 123.95 | 502 | 18.514 | 0.301 | viii |
| | bromo- | | | | | | | |
| 49 | Ethanol, 2- | C6H14O2 | 111-76-2 | 118.1 | 674 | 29.105 | 0.260 | i |
| | butoxy- | | | | | | | |
| 50 | Ethyl Acetate | C4H8O2 | 141-78-6 | 88.052 | 557 | 9.709 | 0.382 | i |
| 51 | Ethylbenzene | C8H10 | 100-41-4 | 106.08 | 823 | 17.156 | 0.434 | ii |
| 52 | Eucalyptol | C10H18O | 470-82-6 | 154.14 | 715 | 20.633 | 0.714 | i |
| 53 | [symbol] | C9H14O | 3777-69-3 | 138.1 | 674 | 21.520 | 0.490 | i |
| 54 | Furan, 3-methyl- | C5H6O | 930-27-8 | 82.042 | 862 | 9.877 | 0.279 | i |
| 55 | Heptadecane | C17H36 | 629-78-7 | 240.28 | 678 | 58.333 | 1.705 | i |
| 56 | [symbol] | C7H14O | 111-71-7 | 114.1 | 762 | 19.733 | 0.698 | i |
| 57 | Hexadecane | C16H34 | 544-76-3 | 226.27 | 657 | 67.200 | 0.682 | i |
| 58 | Hexanal | C6H12O | 66-25-1 | 100.09 | 773 | 15.538 | 0.623 | ii |
| 59 | Hexanal, 2- | C8H16O | 123-05-7 | 128.12 | 657 | 19.742 | 0.873 | i |
| | ethyl- | | | | | | | |
| 60 | Hexane, 2- | C6H13Cl | 638-28-8 | 120.07 | 685 | 6.867 | 0.236 | i |
| | chloro- | | | | | | | |
| 61 | Isoprene | C5H8 | 78-79-5 | 68.063 | 937 | 7.077 | 0.220 | i |
| 62 | Levomenthol | C10H20O | 2216-51-5 | 156.15 | 850 | 38.943 | 0.325 | i |
| 63 | Methyl Isobutyl | C6H12O | 108-10-1 | 100.09 | 627 | 12.923 | 0.663 | i |
| | Ketone | | | | | | | |
| 64 | Methyl vinyl | C4H6O | 78-94-4 | 70.042 | 811 | 11.193 | 0.373 | i |
| | ketone | | | | | | | |
| 65 | n-Hexane | C6H14 | 110-54-3 | 86.11 | 680 | 6.903 | 0.244 | I, ix |
| 66 | [symbol] | C9H18O | 124-19-6 | 142.14 | 839 | 28.833 | 0.730 | ii |
| 67 | Octacosane | C28H58 | 630-02-4 | 394.45 | 779 | 69.751 | 0.670 | i |
| 68 | Octadecane | C18H38 | 593-45-3 | 254.3 | 605 | 20.213 | 1.331 | i |
| 69 | [symbol] | C8H16O | 124-13-0 | 128.12 | 804 | 24.269 | 0.732 | I, ix |
| 70 | Octane | C8H18 | 111-65-9 | 114.14 | 721 | 8.247 | 0.476 | i |
| 71 | Octane, 4- | C8H17Cl | 999-07-5 | 148.1 | 795 | 8.845 | 0.461 | viii |
| | chloro- | | | | | | | |
| 72 | Octane, 4- | C9H20 | 2216-34-4 | 128.16 | 688 | 7.100 | 0.261 | i |
| | methyl- | | | | | | | |
| 73 | Octanoic acid | C8H16O2 | 124-07-2 | 144.12 | 505 | 46.367 | 0.089 | i |
| 74 | o-Cymene | C10H14 | 527-84-4 | 134.11 | 705 | 23.394 | 0.493 | i |
| 75 | Pentacosane | C25H52 | 629-99-2 | 352.41 | 678 | 73.570 | 0.662 | i |
| 76 | Pentanal | C5H10O | 110-62-3 | 86.073 | 585 | 12.080 | 0.517 | i |
| 77 | Pentane, 2,3,4- | C8H18 | 565-75-3 | 114.14 | 711 | 7.791 | 0.433 | i |
| | trimethyl- | | | | | | | |
| 78 | Phenol | C6H6O | 108-95-2 | 94.042 | 792 | 52.287 | 0.057 | iv |
| 79 | Phytol | C20H40O | 150-86-7 | 296.31 | 619 | 37.436 | 0.985 | vii |
| 80 | Propanoic acid | C3H6O2 | 79-09-4 | 74.037 | 775 | 34.972 | 0.087 | V |
| 81 | p-Xylene | C8H10 | 106-42-3 | 106.08 | 791 | 17.510 | 0.441 | i |
| 82 | Styrene | C8H8 | 100-42-5 | 104.06 | 820 | 22.950 | 0.349 | ii, iii |
| 83 | Tetrahydrofuran | C4H8O | 109-99-9 | 72.058 | 642 | 9.284 | 0.411 | ii |
| 84 | Toluene | C7H8 | 108-88-3 | 92.063 | 926 | 14.024 | 0.376 | i |
| 85 | [symbol] | C13H28 | 629-50-5 | 184.22 | 801 | 24.704 | 1.111 | i |
| 86 | Undecane | C11H24 | 1120-21-4 | 156.19 | 713 | 15.589 | 1.120 | i |
| 87 | Undecane, 5- | C12H26 | 1632-70-8 | 170.2 | 517 | 17.500 | 1.259 | i |
| | methyl- | | | | | | | |

Five COVID-19 breath biomarkers were identified with volcano plot, and another VOC approached statistical significance and was also used to diagnose the disease.

Hierarchical Clustering on Principal Components (HCPC) was applied to classify samples using 6 COVID-19 biomarkers. The algorithm of the HCPC method, was implemented in the FactoMineR package, can be summarized as follow: 1) compute principal component method, at this step the number of dimensions to be retained in the output is chosen (ncp=3), 2) compute hierarchical clustering: it is performed using the Ward's criterion on the selected principal components. Ward criterion is used in the hierarchical clustering because it is based on the multidimensional variance like principal component analysis. 3) Choose the number of clusters based on the hierarchical tree (n=2).

### Quality Control

Breath concentration of the canonical human volatile isoprene was performed to quality control for correct breath sampling, as a small or missing isoprene peak indicates an error in the sample collection and/or analysis, resulting in data being excluded. To check for changes in instrument sensitivity over time, a mixture of external standards was analyzed with the GCxGC BenchTOF-MS alongside the breath samples as described previously1. Briefly, we analyzed an external standard before running each batch of breath samples. The standard used was EPA 8240B Calibration Mix (2-butanone, isobutanol, 4-methyl-2-pentanone and 2-hexanone). One mL 2000 μg·mL-1 vial standards were purchased from Sigma-Aldrich. To spike the mixture into a sorbent tube, a 100 μg·mL-1 solution was prepared in HPLC grade methanol. Using a solution loading rig (Markes International Limited, UK), 1 μL of the solution was spiked into a sorbent tube. The sorbent tube was flushed for 3 min with nitrogen at a flow of 100 mL·min-1 and analyzed by GCxGC BenchTOF-MS.

The following example is provided to illustrate certain embodiments of the invention. It is not intended to limit the invention in any way.

### Example I

Children with SARS-CoV-2 infection typically experience mild symptoms of disease and are much less likely to experience severe outcomes, such as hospitalization or death, as compared to adults. Children also exhibit a distinct immunological response to coronavirus infection. These clinical and immunological differences in children with SARS-CoV-2 suggest that their metabolic response to infection may also be different than that of adults. Asymptomatic or mildly symptomatic pediatric cases may transmit SARS-CoV-2 within the household or community. While adults are inconvenienced by social distancing measures to control viral transmission, the educational and social development of children may be irreparably harmed. Finally, until global control of COVID-19 is achieved, children will continue to require a disproportionately high frequency of testing, due to both the burden of clinically indistinguishable non-SARS-CoV-2 upper respiratory viral infections in childhood (up to 12 per year) and the delayed availability of vaccination for young children

SARS-CoV-2 infection is diagnosed through detection of specific viral nucleic acid or antigens from respiratory samples. These techniques are relatively expensive, slow, and susceptible to false-negative results. A rapid noninvasive method to detect infection would be highly advantageous. Compelling evidence from canine biosensors and studies of adults with COVID-19 suggests that infection reproducibly alters human volatile organic compound (VOC) profiles. To determine whether pediatric infection is associated with VOC changes, we enrolled SARS-CoV-2 infected and uninfected children admitted to a major pediatric academic medical center. Breath samples were collected from children and analyzed through state-of-the-art GCxGC-ToFMS. Isolated features included 84 targeted VOCs. Candidate biomarkers that were correlated with infection status were subsequently validated in a second, independent cohort of children. We thus find that six volatile organic compounds are significantly and reproducibly increased in the breath of SARS-CoV-2 infected children. Three aldehydes (octanal, nonanal, and heptanal) drew special attention, as aldehydes are also elevated in the breath of adults with COVID-19. Together, these biomarkers demonstrate high accuracy for distinguishing pediatric SARS-CoV-2 infection and support the ongoing development of novel breath-based diagnostics.

Metabolic changes induced by respiratory infections may alter host odor profiles, such that infection-associated volatile organic compounds (VOCs) may be used to develop noninvasive diagnostics through sensor arrays (e.g., “breath-alyzers”) or electronic noses. Promising proof-of-concept comes from studies of other respiratory infections that lead to characteristic alterations in breath metabolite profiles, including infection with Mycobacterium tuberculosis⁷ and Aspergillus spp., as well as ventilator-associated pneumonia.⁸ Viral respiratory pathogens impact host volatile production in vitro and in vivo. For example, infection with either rhinovirus⁹ or influenza in cell culture¹⁰ results in reproducible VOC changes. Similarly, studies in an animal model of influenza infection demonstrate an increase in breath concentrations of acetaldehyde, propanal, and n-propyl acetate.¹¹ More recently, compelling evidence from canine biosensors suggests that volatile detection may be a promising approach for SARS-CoV-2 diagnosis. Trained dogs reproducibly recognize SARS-CoV-2 infection in saliva/tracheal samples,¹² urine,¹³ and sweat samples.¹⁴ In addition, distinct breath signatures were found in adult patients with COVID-19, compared to those with unrelated respiratory and cardiac conditions.¹⁵ Preliminary studies using sensor arrays confirm that SARS-CoV-2 infection in adults likewise results in distinct breath volatile changes.¹⁶

To evaluate whether changes in breath metabolites also characterize the exhaled breath of pediatric patients with SARS-CoV-2 infection, we analyzed breath metabolite profiles from two independent cohorts of children with and without SARS-CoV-2, who were admitted to a major pediatric academic medical center (for workflow, see FIG. 2). Through targeted GCxGC-mass spectrometric analysis of 84 breath volatiles (Table 1A), we established candidate biomarkers of pediatric SARS-CoV-2 infection, which were validated in an independent cohort of children.

Biomarker discovery was performed from breath metabolic profiling of pediatric patients (n=26) from the Children's Hospital of Philadelphia (CHOP), 11 of whom were positive and 15 of whom were negative for SARS-CoV-2 by nasopharyngeal (NP) RT-PCR. One SARS-CoV-2 infected subject was excluded due to poor quality of breath sampling.

Demographic and clinical characteristics in this discovery cohort are shown in Table 1B. SARS-CoV-2 infected and uninfected patients were broadly similar with respect to age, sex, and racial/ethnic characteristics. Individuals infected with SARS-CoV-2 were more likely to exhibit either fever (50% vs 0.0%, p=0.004) or cough (40% vs 0.0%, p=0.016), compared to uninfected subjects. Two SARS-CoV-2 positive subjects (25%) lacked symptoms of acute infection (specifically fever, sore throat, cough, or GI symptoms). Two subjects with positive nasopharyngeal testing for SARS-CoV-2 were subsequently diagnosed with multisystem inflammatory syndrome in children (MIS-C), believed to be a late complication of SARS-CoV-2 infection.

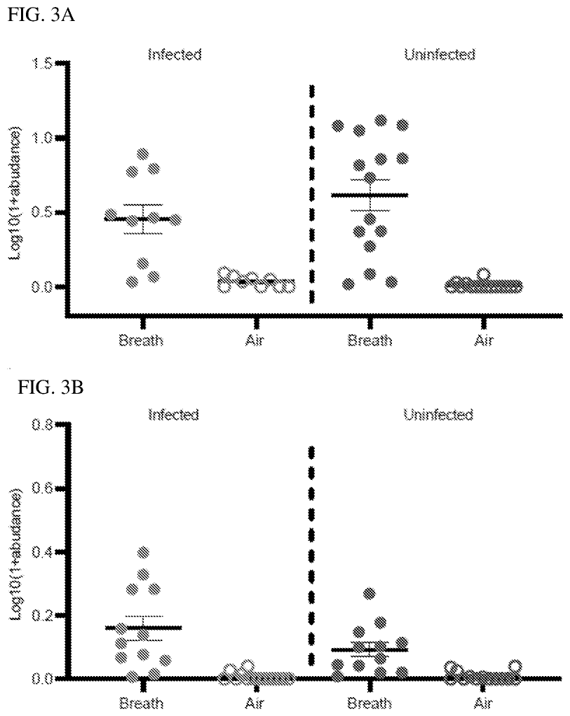

For each patient, breath volatiles were captured onto sorbent material and subsequently released by thermal desorption for analysis by two-dimensional gas chromatography and time-of-flight mass spectrometry (GCxGC ToF-MS). Isoprene is one of the most common and abundant human breath VOCs. To establish the quality of breath VOC collection, the abundance of isoprene was compared to the abundance of isoprene in ambient air that had been collected in the same room and at the same time as breath collection. For each subject, we find that the abundance of isoprene was markedly higher than ambient levels, confirming successful breath VOC collection (FIGS. 3A-3B).

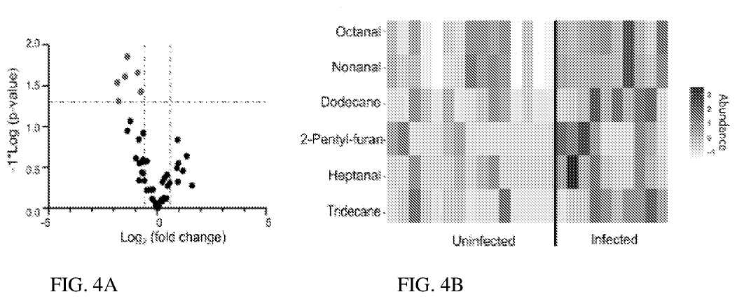

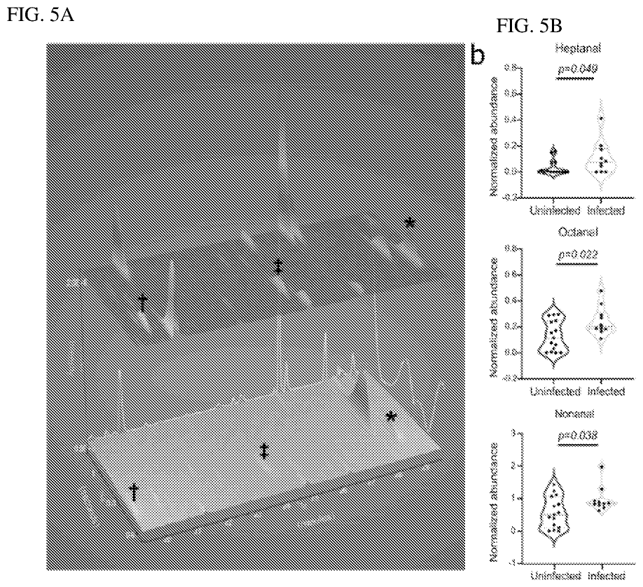

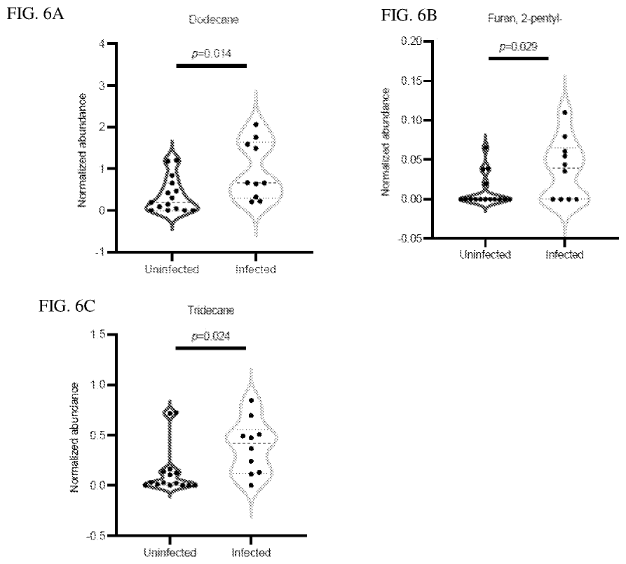

For our targeted metabolite analysis, we selected 84 VOCs that have previously been identified as either common human odorants¹⁷ or ones associated with host response to viral infection or were found to be elevated in the breath of adults with COVID-19.^{9-11,15,18,19} Volcano plots (FIG. 4A) were used to visualize breath metabolic features that distinguished SARS-CoV-2 infected from uninfected individuals, using p<0.05 as a threshold for statistical significance. Six candidate breath biomarkers were significantly elevated in the breath of children with SARS-CoV-2 infection: three aldehydes [octanal, nonanal, and heptanal (FIGS. 5A-5B)], as well as decane, tridecane, and 2-pentyl furan (FIG. 4B and FIG. 6). All compound identities were confirmed by comparison to pure commercial standards. Analytical characteristics of candidate breath biomarkers can be found in FIG. 1B.

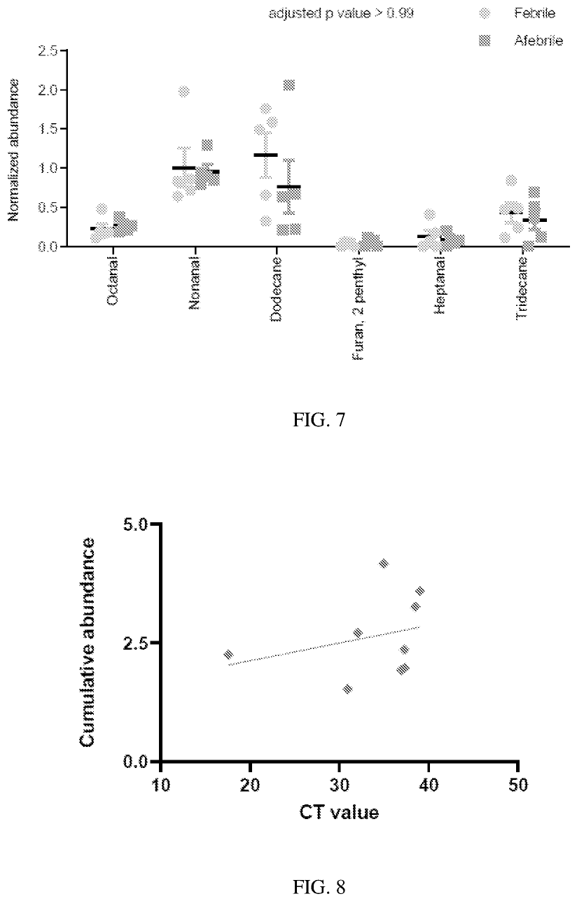

Heat map visualization indicates an increase in the abundance of candidate volatile biomarkers in the breath of children with SARS-CoV-2 infection (FIG. 4B), suggesting that SARS-CoV-2 infection alters the overall profile of breath VOCs. Because elevated temperature alone can alter metabolic profiles, we evaluated whether any candidate biomarkers correlated with fever. We find that fever was not associated with significant changes in abundance of any SARS-CoV-2 biomarker (FIG. 7). In addition, we found no correlation between specific VOC concentrations and the viral load [as approximated by qRT-PCR cycle threshold (Ct) value], suggesting that these volatiles are markers of host response rather than virus carriage (FIG. 8).

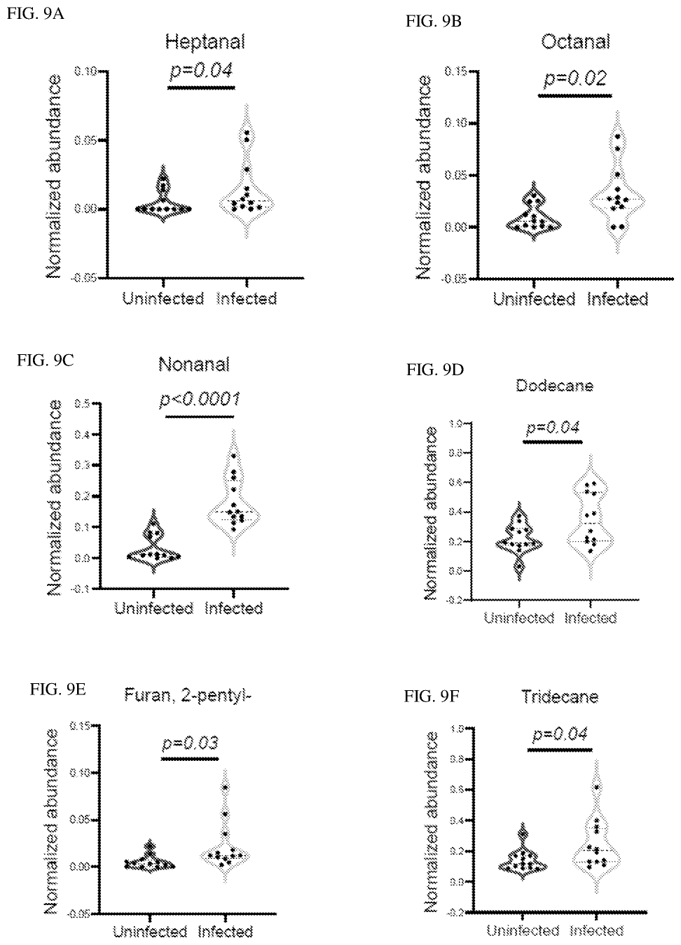

To establish the reproducibility of these candidate biomarkers, independent subjects were enrolled in a validation cohort of children with and without SARS-CoV-2 infection (n=24). Patients enrolled had similar demographic and clinical characteristics as the discovery cohort, and infected and uninfected children were broadly similar (Table 1B). We found that all candidate SARS-CoV-2 biomarkers were increased in abundance in infected compared to uninfected children in this validation cohort (FIG. 9). To visualize the discriminatory

TABLE 1B
Patient Demographics and Clinical Characteristics

| | discovery cohort | | | validation cohort | | |
|---|---|---|---|---|---|---|
| | SARS-Cov-2 | SARS-COV-2 | | SARS-COV-2 | SARS-COV-2 | |
| variables | negative (n = 15) | positive (n = 10) | p value | negative (n = 12) | positive (n = 12) | p value |

Demographic Characteristics

| | | | | | | | | | | |
|---|---|---|---|---|---|---|---|---|---|---|
| age (years), median (IQR) female, | 15 | (12-16) | 11 | (8.2-17) | 0.15 | 15 | (14-16) | 12.5 | (9.25-15.75) | 0.022 |
| n (%) | 9 | (60) | 6 | (60) | >0.996^{b} | 9 | (75) | 6 | (50) | >0.40^{b} |
| black or African-American, n (%) | 6 | (40) | 5 | (50) | 0.69^{b} | 5 | (42) | 4 | (33) | >0.99^{b} |
| BMI/age percentile, median (IQR) | 86 | (62.5-98)^{c} | 61.5 | (45-84) | 0.11 | 26 | (21-31) | 19 | (17-26)^{d} | 0.09 |

Reported Symptoms, n (%)

| | | | | | | | | | | |
|---|---|---|---|---|---|---|---|---|---|---|
| fever (>38.0° C.) | 0 | (0) | 5 | (50) | 0.004^{b} | 0 | (0) | 2 | (17) | 0.47^{b} |
| cough (new onset or worsening of chronic cough) | 0 | (0) | 4 | (40) | 0.016^{b} | 0 | (0) | 0 | (0) | >0.99^{b} |
| sore throat | 0 | (0) | 1 | (10) | 0.40^{b} | 0 | (0) | 1 | (8) | >0.99^{b} |
| headache | 0 | (0) | 1 | (10) | 0.40^{b} | 0 | (0) | 1 | (8) | >0.99^{b} |

Laboratory

| | | | | |
|---|---|---|---|---|
| cyde threshold values (SARS-COV-2 RT- | >40 | 36.74 (31.78- | >40 | 28.83 (22.15- |
| PCR), medien (IQR) | (negative) | 37.63) | (negative) | 32.92)^{d} |

^{a}Data represent median value (interquartile range) or number of patients (%).
^{b}Fisher's exact test used for contingency table analysis.
^{c}Data unavailable for two patients.
^{d}Data unavailable for one patient.
^{e}Data unavailable for four patients.

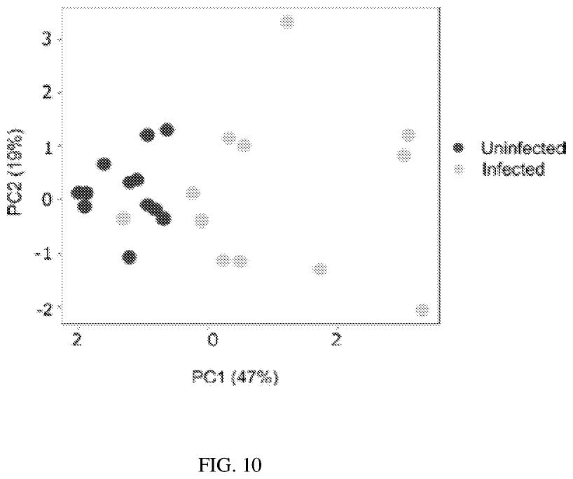

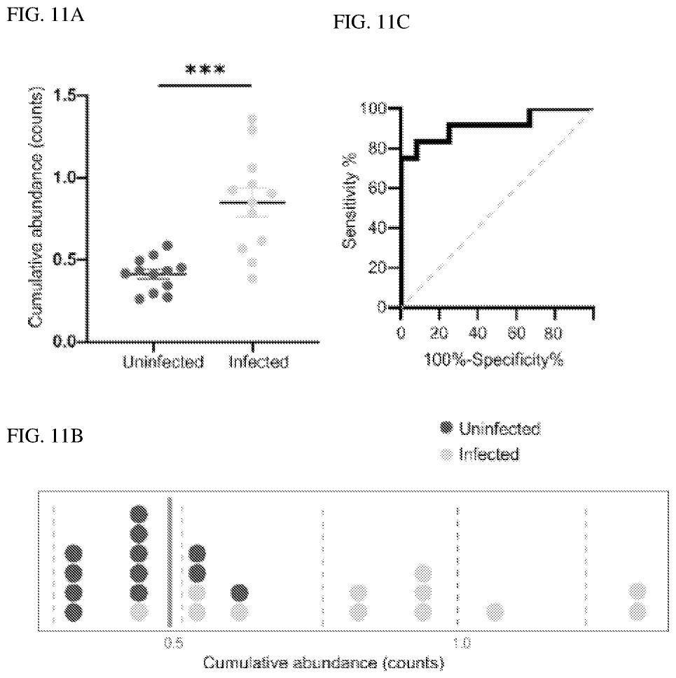

power of these biomarkers, principal components analysis (PCA) was performed (FIG. 10). This technique indicates substantial differences in breath volatile composition in the validation cohort between SARS-CoV-2 infected children and children without infection. Using the sum of the abundances of these 6 biomarkers (“cumulative abundance”) as a diagnostic strategy, we evaluated its diagnostic characteristics. A receiver operating characteristic (ROC) curve yielded an area under the curve (AUC) of 0.92, providing a sensitivity of 91% and specificity of 75% (FIG. 11A, 11B, 11C). Thus, our breath biomarkers approach the WHO priority product profile for COVID-19 diagnostics (“acceptable” sensitivity ≥80% and specificity ≥97%) for point-of-care testing, for suspected COVID-19 cases where RT-PCR cannot be delivered in a timely manner.²⁰ Of note, lower specificities are appropriate for population-based screening tests (e.g., in airports or prior to large unmasked indoor events), because they are not used for clinical decision-making and positive results will be secondarily validated by molecular testing. Overall, our results suggest that SARS-CoV-2 infection leads to characteristic and reproducible changes in breath volatiles in children and that as few as 6 volatiles may be used to diagnose SARS-CoV-2 with high accuracy.

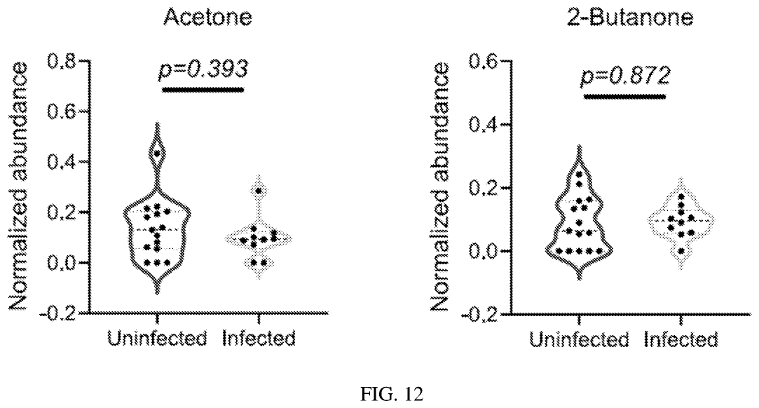

Compared to adults, children have a distinct immune response and are less likely to become seriously ill with SARS-CoV-2 infection. For this reason, biomarkers enriched in the breath of adults with symptomatic COVID-19 may be distinct from those in children. We investigated whether breath volatiles that were previously found to be associated with adult COVID-19 were also present in our pediatric samples.¹⁵ We find that two medium-chain aldehydes that are elevated in the breath of adults with COVID-19, octanal and heptanal, are also elevated in the breath of children with SARS-CoV-2 infection (FIG. 5B). Interestingly, pediatric SARS-CoV-2 infection was also associated with increased levels of a third aldehyde, nonanal. In contrast, two chemically distinct breath biomarkers of SARS-CoV-2 infection in adults, acetone and 2-butanone, are not significantly altered in SARS-CoV-2 infected children compared to uninfected controls (FIG. 12).

Frequent, rapid testing has been proposed as an important public health strategy for control of the current COVID-19 pandemic. An easy-to-use SARS-CoV-2 “breathalyzer” based on electronic nose technologies or sensor arrays would have a turnaround time of minutes and would not strain supply chains of specialized disposable supplies. Because of ongoing advances in portable, low-cost, field-stable sensor array platforms that may be harnessed for a VOC-based diagnostic, there is enthusiastic industry support that may rapidly translate volatile biomarkers into physical devices for point-of-care testing or screening.

Strong evidence indicates that SARS-CoV-2 infection in adults leads to a unique human odorant profile. Canine biosensors (sniffer dogs) accurately recognize SARS-CoV-2 infection in biological samples and have begun to be deployed for human screening in real-world settings such as airports and sports arenas.^{12,14} Previous studies by Ruszkiewicz et al.¹⁵ also report breath biomarkers associated with COVID-19 in adults presenting for emergency room evaluation with acute respiratory symptoms. They find that breath biomarkers distinguished patients with COVID-19 from those with other respiratory conditions with high (>80%) accuracy.¹⁵ Metabolites, including breath volatile organic compounds, that are associated with viral infection are most likely to arise from changes in host metabolism, raising the possibility that populations that differ in their clinical response to infection might also have divergent metabolic profiles. Since pediatric SARS-CoV-2 infection is generally mild and less likely to result in serious respiratory symptoms, we expected that the breath metabolic biomarkers of children infected with SARS-CoV-2 might differ from those found in adults. In this work, we provide compelling support that SARS-CoV-2 infection leads to characteristic volatile organic compound changes in the breath of children, as it does in adults. However, we find that the breath volatile signature of pediatric SARS-CoV-2 is distinct. For example, pediatric SARS-CoV-2 infection does not lead to changes in some specific breath biomarkers, such as acetone and 2-butanone, that are highly characteristic of COVID-19 in adults.

This work provides important validation that SARS-CoV-2 infection leads to changes in breath aldehyde concentrations in both children and adults. The prior study by Ruszkiewicz et al. found that increased levels of two aldehydes, octanal and heptanal, were present in the breath of adults with SARS-CoV-2 infection, compared to those with other acute respiratory illnesses (such as COPD and pneumonia). We find that octanal, heptanal, and the structurally similar aldehyde nonanal are all significantly elevated in the breath of children with SARS-CoV-2 infection.

Increasing evidence suggests that SARS-CoV-2 infection in adults is associated with specific changes in volatile production. This study provides additional support that the breath abundance of six volatile organic compounds (including aldehydes) are altered in children with SARS-CoV2 infection. Importantly, most SARS-CoV-2 infected subjects enrolled demonstrated mild symptoms of infection and were only incidentally found to be infected due to routine preadmission screening at our institution. Given the cost, discomfort, and false-negative results associated with RT-PCR- or antigen-based tests, breathalyzer testing for SARS-CoV-2 provides an inexpensive, noninvasive, rapid, and highly sensitive alternative for population-based frequent screening of large numbers of individuals (e.g., screening at airports or before attending large indoor events). In the case of screening, performance characteristics are less important than ease and rapidity of testing, since positive results can be verified by secondary, more specific tests.

### REFERENCES

(1) Wang, Y., Kang, H., Liu, X., and Tong, Z. (2020) Combination of RT-qPCR testing and clinical features for diagnosis of COVID-19 facilitates management of SARS-CoV-2 outbreak. J. Med. Virol. 92, 538-539.
(2) Li, X., Xu, W., Dozier, M., He, Y., Kirolos, A., Theodoratou, E., and UNCOVER (2020) The role of children in transmission of SARS-CoV-2: A rapid review. J. Glob Health 10, 011101.
(3) Diorio, C., Henrickson, S. E., Vella, L. A., McNerney, K. O., Chase, J., Burudpakdee, C., Lee, J. H., Jasen, C., Balamuth, F., Barrett, D. M., Banwell, B. L., Bernt, K. M., Blatz, A. M., Chiotos, K., Fisher, B. T., Fitzgerald, J. C., Gerber, J. S., Gollomp, K., Gray, C., Grupp, S. A., Harris, R. M., Kilbaugh, T. J., John, A. R. O., Lambert, M., Liebling, E. J., Paessler, M. E., Petrosa, W., Phillips, C., Reilly, A. F., Romberg, N. D., Seif, A., Sesok-Pizzini, D. A., Sullivan, K. E., Vardaro, J., Behrens, E. M., Teachey, D. T., and Bassiri, H. (2020) Multisystem inflammatory syndrome in children and COVID-19 are distinct presentations of SARS-CoV-2. The. J. Clin. Invest. 130, 5967-5975.
(4) Weisberg, S. P., Connors, T. J., Zhu, Y., Baldwin, M. R., Lin, W. H., Wontakal, S., Szabo, P. A., Wells, S. B., Dogra, P., Gray, J., Idzikowski, E., Stelitano, D., Bovier, F. T., Davis-Porada, J., Matsumoto, R., Poon, M. M. L., Chait, M., Mathieu, C., Horvat, B., Decimo, D., Hudson, K. E., Zotti, F. D., Bitan, Z. C., La Carpia, F., Ferrara, S. A., Mace, E., Milner, J., Moscona, A., Hod, E., Porotto, M., and Farber, D. L. (2021) Distinct antibody responses to SARS-CoV-2 in children and adults across the COVID-19 clinical spectrum. Nat. Immunol. 22, 25-31.
(5) Schwartz, N. G., Moorman, A. C., Makaretz, A., Chang, K., Chu, V. T., Szablewski, C. M., Yousaf, A. R., Brown, M. M., Clyne, A., DellaGrotta, A., Drobeniuc, J., Korpics, J., Muir, A., Drenzek, C., Bandy, U., Kirking, H. L., Tate, J. E., Hall, A. J., Lanzieri, T. M., and Stewart, R. J. (2020) Adolescent with COVID-19 as the Source of an Outbreak at a 3-Week Family Gathering—Four States. MMWR Morb Mortal Wkly Report 69, 1457-1459.
(6) Nokso-Koivisto, J., Pitkaranta, A., Blomqvist, S., Jokinen, J., Kleemola, M., Takala, A., Kilpi, T., and Hovi, T. (2002) Viral etiology of frequently recurring respiratory tract infections in children. Clin. Infect. Dis. 35, 540-6.
(7) Phillips, M., Basa-Dalay, V., Blais, J., Bothamley, G., Chaturvedi, A., Modi, K. D., Pandya, M., Natividad, M. P. R., Patel, U., Ramraje, N. N., Schmitt, P., and Udwadia, Z. F. (2012) Point-of-care breath test for biomarkers of active pulmonary tuberculosis. Tuberculosis 92, 314-320.
(8) Filipiak, W., Beer, R., Sponring, A., Filipiak, A., Ager, C., Schiefecker, A., Lanthaler, S., Helbok, R., Nagl, M., Troppmair, J., and Amann, A. (2015) Breath analysis for in vivo detection of pathogens related to ventilator-associated pneumonia in intensive care patients: a prospective pilot study. J. Breath Res. 9, 016004.
(9) Schivo, M., Aksenov, A. A., Linderholm, A. L., McCartney, M. M., Simmons, J., Harper, R. W., and Davis, C. E. (2014) Volatile emanations from in vitro airway cells infected with human rhinovirus. J. Breath Res. 8, 037110.
(10) Purcaro, G., Rees, C. A., Wieland-Alter, W. F., Schneider, M. J., Wang, X., Stefanuto, P. H., Wright, P. F., Enelow, R. I., and Hill, J. E. (2018) Volatile fingerprinting of human respiratory viruses from cell culture. J. Breath Res. 12, 026015.
(11) Traxler, S., Bischoff, A. C., Sass, R., Trefz, P., Gierschner, P., Brock, B., Schwaiger, T., Karte, C., Blohm, U., Schroder, C., Miekisch, W., and Schubert, J. K. (2018) VOC breath profile in spontaneously breathing awake swine during Influenza A infection. Sci. Rep. 8, 14857.
(12) Jendrny, P., Schulz, C., Twele, F., Meller, S., von Kockritz-Blickwede, M., Osterhaus, A., Ebbers, J., Pilchova, V., Pink, I., Welte, T., Manns, M. P., Fathi, A., Ernst, C., Addo, M. M., Schalke, E., and Volk, H. A. (2020) Scent dog identification of samples from COVID-19 patients—a pilot study. BMC Infect. Dis. 20, 536.
(13) Essler, J. L., Kane, S. A., Nolan, P., Akaho, E. H., Berna, A. Z., DeAngelo, A., Berk, R. A., Kaynaroglu, P., Plymouth, V. L., Frank, I. D., Weiss, S. R., Odom John, A. R., and Otto, C. M. (2021) Discrimination of SARS-CoV-2 infected patient samples by detection dogs: A proof of concept study. PLoS One 16, e0250158.
(14) Grandjean, D., et al. Detection dogs as a help in the detection of COVID-19 Can the dog alert on COVID-19 positive persons by sniffing axillary sweat samples? Proof-of-concept study. bioRxiv 2020, see the world wide web at biorxiv.org/content/10. 1101/2020.06.03.132134v1.
(15) Ruszkiewicz, D. M., Sanders, D., O'Brien, R., Hempel, F., Reed, M. J., Riepe, A. C., Bailie, K., Brodrick, E., Darnley, K., Ellerkmann, R., Mueller, O., Skarysz, A., Truss, M., Wortelmann, T., Yordanov, S., Thomas, C. L. P., Schaaf, B., and Eddleston, M. (2020) Diagnosis of COVID-19 by analysis of breath with gas chromatography-ion mobility spectrometry—a feasibility study. EClinicalMedicine 29, 100609.
(16) Shan, B., Broza, Y. Y., Li, W., Wang, Y., Wu, S., Liu, Z., Wang, J., Gui, S., Wang, L., Zhang, Z., Liu, W., Zhou, S., Jin, W., Zhang, Q., Hu, D., Lin, L., Zhang, Q., Li, W., Wang, J., Liu, H., Pan, Y., and Haick, H. (2020) Multiplexed Nanomaterial-Based Sensor Array for Detection of COVID-19 in Exhaled Breath. ACS Nano 14, 12125-12132.
(17) de Lacy Costello, B. D., Amann, A., Al-Kateb, H., Flynn, C., Filipiak, W., Khalid, T., Osborne, D., and Ratcliffe, N. M. (2014) A review of the volatiles from the healthy human body. J. Breath Res. 8, 014001.
(18) Aksenov, A. A., Sandrock, C. E., Zhao, W., Sankaran, S., Schivo, M., Harper, R., Cardona, C. J., Xing, Z., and Davis, C. E. (2014) Cellular scent of influenza virus infection. ChemBioChem 15, 1040-8.
(19) Traxler, S., Barkowsky, G., Sass, R., Klemenz, A. C., Patenge, N., Kreikemeyer, B., Schubert, J. K., and Miekisch, W. (2019) Volatile scents of influenza A and S. pyogenes (co-)infected cells. Sci. Rep. 9, 18894.
(20) World Health Organization Target product profiles for priority diagnostics to support response to the COVID-19 pandemic v.1.0; World Health Organization: 2020.
(21) Schwarz, K. B. (1996) Oxidative stress during viral infection: a review. Free Radical Biol. Med. 21, 641-9.
(22) Ratcliffe, N., Wieczorek, T., Drabinska, N., Gould, O., Osborne, A., and De Lacy Costello, B. (2020) A mechanistic study and review of volatile products from peroxidation of unsaturated fatty acids: an aid to understanding the origins of volatile organic compounds from the human body. J. Breath Res. 14, 034001.
(23) Ayala, A., Munoz, M. F., and Arguelles, S. (2014) Lipid peroxidation: production, metabolism, and signaling mechanisms of malondialdehyde and 4-hydroxy-2-nonenal. Oxid. Med. Cell. Longevity 2014, 360438.
(24) Yan, B., Chu, H., Yang, D., Sze, K. H., Lai, P. M., Yuan, S., Shuai, H., Wang, Y., Kao, R. Y., Chan, J. F., and Yuen, K. Y. (2019) Characterization of the Lipidomic Profile of Human Coronavirus-Infected Cells: Implications for Lipid Metabolism Remodeling upon Coronavirus Replication. Viruses 11, 73.
(25) Al-Kateb, H., Cunliffe, N., deLacy Costello, B., Probert, C., and Ratcliffe, N. (2012) PWE-169 Analysis of faecal volatiles from young children infected with and without rotavirus. Gut 61, A364.3.
(26) Berna, A. Z., Debosch, B., Stoll, J., and Odom John, A. R. (2019) Breath Collection from Children for Disease Biomarker Discovery. J. Visualized Exp., e59217.
(27) Berna, A. Z., McCarthy, J. S., Wang, X. R., Michie, M., Bravo, F. G., Cassells, J., and Trowell, S. C. (2018) Diurnal variation in expired breath volatiles in malaria-infected and healthy volunteers. J. Breath Res. 12, 046014.
(28) Berna, A. Z., McCarthy, J. S., Wang, R. X., Saliba, K. J., Bravo, F. G., Cassells, J., Padovan, B., and Trowell, S. C. (2015) Analysis of breath specimens for biomarkers of Plasmodium falciparum infection. J. Infect. Dis. 212, 1120-1128.

### REFERENCES FOR VOLATILES IN TABLE 1A

i de Lacy Costello, B., et al., A review of the volatiles from the healthy human body. J Breath Res, 2014. 8(1): p. 014001.
ii Purcaro, G., et al., Volatile fingerprinting of human respiratory viruses from cell culture. J Breath Res, 2018. 12(2): p. 026015.
iii Traxler, S., et al., VOC breath profile in spontaneously breathing awake swine during Influenza A infection. Sci Rep, 2018. 8(1): p. 14857.
iv Schivo, M., et al., Volatile emanations from in vitro airway cells infected with human rhinovirus. J Breath Res, 2014. 8(3): p. 037110.
v Aksenov, A. A., et al., Cellular scent of influenza virus infection. Chembiochem, 2014. 15(7): p. 1040-8
vi Traxler, S., et al., Volatile scents of influenza A and S. pyogenes (co-)infected cells. Scientific Reports, 2019. 9.
vii Zhang, A. Q., S. C. Mitchell, and R. L. Smith, Dimethylamine in human urine. Clin Chim Acta, 1995. 233(1-2): p. 81-8
viii Unpublished authors' own breath library
ix Al-Kateb, H. Analysis of faecal volatiles from young children infected with or without Rotavirus. 2012.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. A method for diagnosing or monitoring a subject with a SARS-COV-2 infection, the method comprising analyzing a sample of exhaled breath or condensate breath obtained from the subject for elevated levels of volatile organic compounds (VOCs) octanal, nonanal, and heptanal relative to levels observed in uninfected control pediatric subjects, wherein said elevated levels are indicative of SARS-COV-2 infection.

2. The method of claim 1, further comprising assessment of decane, 2-penthyl furan, heptanal, tridecane and dodecane levels.

3. The method claim 1 wherein analysis of the VOC comprises the use of at least one technique selected from the group consisting of photo ionization detection, flame ionization detection, gas chromatography—mass spectrometry (GC-MS), proton transfer reaction mass spectrometry (PTR-MS), colorimetry, infrared spectroscopy, electrochemical fuel cell sensing, semiconductor gas sensing, quartz tuning fork (QTF) sensors, electronic noses and combinations thereof.

4. The method of claim 1 wherein analysis of the VOCs is conducted using a portable, hand-held breathalyzer device.

5. The method of claim 1 comprising: analyzing the sample of exhaled breath or condensate breath obtained from the subject for a series of volatile organic compounds (VOCs) comprising:
i) octanal;
ii) nonanal;
iii heptanal;
iv) decane;
v) 2-penthyl furan; and
vi tridecane
determining a concentration for each of the VOCs; and
calculating a cumulative abundance based on the concentrations for the VOCs, wherein the cumulative abundance and the concentration of the VOCs indicates a SARS-COV-2 infection, wherein said subject is a pediatric, adolescent or adult subject.

6. A method for diagnosing or monitoring a subject with a SARS-COV-2 infection, comprising: analyzing a sample of exhaled breath or condensate breath obtained from the subject for a series of volatile organic compounds (VOCs) comprising:
i) octanal;
ii) nonanal;
iii) heptanal;
iv) decane;
v) 2-penthyl furan; and
vi) tridecane
determining a concentration for each of the VOCs; and
calculating a cumulative abundance based on the concentrations for the VOCs, wherein the cumulative abundance indicates a SARS-Co-V-2 infection.

7. The method of claim 6 wherein the analysis of the series of VOCs comprises the use of at least one technique selected from the group consisting of photo ionization detection, flame ionization detection, gas chromatography—mass spectrometry (GC-MS), proton transfer reaction mass spectrometry (PTR-MS), colorimetry, infrared spectroscopy, electrochemical fuel cell sensing, semiconductor gas sensing, quartz tuning fork (QTF) sensors, electronic noses and combinations thereof.

8. The method of claim 6 wherein the analysis of the series of VOCs comprises is conducted using a portable, hand-held breathalyzer device.

9. The method of claim 1 wherein the sample is exhaled breath.

10.A method of detecting a combination of VOCs in a subject selected from octanal, nonanal, heptanal, decane or dodecane, 2-penthyl furan; and tridecane, the method comprising analyzing a sample of exhaled breath or condensate breath obtained from the subject for said VOCs.

11. The method of claim 10, wherein analysis of the at least one monoterpene comprises the use of at least one technique selected from the group consisting of photo ionization detection, flame ionization detection, gas chromatography - mass spectrometry (GC-MS), proton transfer reaction mass spectrometry (PTR-MS), colorimetry, infrared spectroscopy, electrochemical fuel cell sensing, semiconductor gas sensing, quartz tuning fork (QTF) sensors, electronic noses and combinations thereof.

12. The method of claim 10 wherein analysis of the VOCs is conducted using a portable, hand-held breathalyzer device.

13. The method of claim 1, further comprising condensing or concentrating the sample before analysis.

14. The method of claim 1, further comprising administering to the subject a pharmaceutical composition comprising a therapeutically effective amount of at least one compound effective against a SARS-COV-2 infection.

15. The method of claim 14 wherein the compound effective against the SARS-CoV-2 infection comprises at least compound selected from the group consisting of convalescent plasma, remdesivir, regeneron, soluble ACE-2 and steroids, and combinations thereof.

16. The method of claim 15, wherein at least two compounds effective against the SARS-COV-2 infection are administered.

17. The method of claim 6, wherein the level of at least one additional markers in Table 1A is determined.

18. The method of claim 6, wherein the levels of all of the markers in Table 1A are determined.

19. The method of claim 10, further comprising condensing or concentrating the sample before analysis.

20. The method of claim 10, further comprising administering to the subject a pharmaceutical composition comprising a therapeutically effective amount of at least one compound effective against a SARS-COV-2 infection.
